(19) United States
(12) Patent Application Publication
(10) Pub. No.: US 2024/0176042 A1
(43) Pub. Date: May 30, 2024

(54) METHODS FOR DIAGENETIC ROCK FACIES CLASSIFICATION AND NODULAR EVAPORITE DEPOSIT CHARACTERIZATION

(71) Applicant: SAUDI ARABIAN OIL COMPANY, Dhahran (SA)

(72) Inventors: Aqeel M. KHALIFA, Dammam (SA); Mandefro B. WOLDEAMANUEL, Dhahran (SA)

(73) Assignee: SAUDI ARABIAN OIL COMPANY, Dhahran (SA)

(21) Appl. No.: 18/060,387

(22) Filed: Nov. 30, 2022

(51) Int. Cl. G01V 11/00; E21B 47/002; E21B 49/00

(52) U.S. Cl. CPC G01V 11/002; E21B 47/002; E21B 49/00

(57) ABSTRACT

A method may include imaging a portion of a subterranean formation to obtain a formation image. The formation image may be analyzed to identify one or more nodular evaporite deposits in the portion of the subterranean formation. One or more deposit characteristics may be determined for each of the one or more nodular evaporite deposits, the one or more deposit characteristics comprising a cross-sectional area, a volume, a roundness index, a vertical depth relative to the subterranean formation, a degree of occlusion, an overlap of deposits, or any combination thereof. The portion of the subterranean formation may be classified into a diagenetic rock facies based on the one or more deposit characteristics of each of the one or more nodular evaporite deposits per unit volume and a number of the one or more nodular evaporite deposits per the unit volume.

### FIELD OF THE DISCLOSURE

The present disclosure relates generally to classifying the diagenetic rock facies of subterranean formations.

### BACKGROUND OF THE DISCLOSURE

Diagenesis refers to the physical and chemical processes that occur from the start of deposition, continuing through compaction, cementation, and dissolution of a rock structure. Cementation, or the formation of cement nodules, can have a significant impact on the quality and heterogeneity of petroleum reservoirs in subterranean formations. Certain characteristics of the cement nodules may be approximated by conventional geochemical logging, but correlations to rock facies is lacking.

### SUMMARY OF THE DISCLOSURE

Various details of the present disclosure are hereinafter summarized to provide a basic understanding. This summary is not an extensive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

According to an embodiment consistent with the present disclosure, a method may comprise imaging a portion of a subterranean formation to obtain a formation image. The formation image may be analyzed to identify one or more nodular evaporite deposits in the portion of the subterranean formation. One or more deposit characteristics may be determined for each of the one or more nodular evaporite deposits, the one or more deposit characteristics comprising a cross-sectional area, a volume, a roundness index, a vertical depth relative to the subterranean formation, a degree of occlusion, an overlap of deposits, or any combination thereof. The portion of the subterranean formation may be classified into a diagenetic rock facies based on the one or more deposit characteristics of each of the one or more nodular evaporite deposits per unit volume and a number of the one or more nodular evaporite deposits per the unit volume.

In another embodiment, a method may comprise imaging a portion of a subterranean formation to obtain a formation image. The formation image may be analyzed to identify one or more nodular evaporite deposits in the portion of the subterranean formation. One or more deposit characteristics may be determined for each of the one or more nodular evaporite deposits, the one or more deposit characteristics comprising a cross-sectional area, a volume, a roundness index, a vertical depth relative to the subterranean formation, a degree of occlusion, an overlap of deposits, or any combination thereof. The portion of the subterranean formation may be classified into a diagenetic rock facies based on the one or more deposit characteristics of each of the one or more nodular evaporite deposits per unit volume and a number of the one or more nodular evaporite deposits per the unit volume. A three-dimensional (3D) model of the portion of the subterranean formation may be generated from the diagenetic rock facies.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

FIG. 1 illustrates a method for diagenetic rock classification.

FIG. 2 illustrates the identification of nodular evaporite deposits from a 360° core photo.

FIG. 3 illustrates the identification of nodular evaporite deposits from a borehole image log.

FIG. 4 is a graph comparing the identification of nodular evaporite deposits from a 360° core photo and a geochemical log.

FIG. 5 illustrates one example of a computer system that can be employed to execute one or more embodiments of the present disclosure.

### DETAILED DESCRIPTION

Embodiments of the present disclosure will now be described in detail with reference to the accompanying Figures. Like elements in the various figures may be denoted by like reference numerals for consistency. Further, in the following detailed description of embodiments of the present disclosure, numerous specific details are set forth in order to provide a more thorough understanding of the claimed subject matter. However, it will be apparent to one of ordinary skill in the art that the embodiments disclosed herein may be practiced without these specific details. In other instances, well-known features have not been described in detail to avoid unnecessarily complicating the description. Additionally, it will be apparent to one of ordinary skill in the art that the scale of the elements presented in the accompanying Figures may vary without departing from the scope of the present disclosure.

Embodiments in accordance with the present disclosure generally relate to classifying the diagenetic rock facies of subterranean formations and, more particularly, to identifying and characterizing nodular evaporite deposits in subterranean formations. The porosity of subterranean rock may be altered through various geological processes, including naturally-occurring diagenetic cement phases, or diagenesis. Such cement phases can vary significantly across formation rocks. The misidentification of cemented rock, or cement nodules, may cause inaccurate calculations of the formation's petroleum storage capacity and permeability.

A novel workflow has been developed to identify, characterize, and quantify one or more individual nodular evaporite deposits in subterranean formations using formation images. Formation images are commonly gathered in subsurface logging methods for stratigraphic or structural information. From the formation images, characteristics such as size, roundness, connectivity of occlusions, degree of occlusion in porosity, and quantity of nodular evaporite deposits may be determined. These characteristics may be used to classify the diagenetic rock facies of the subterranean formation, assisting in the accurate calculation of the formation's capacity and permeability.

The terminology used herein is for the purpose of describing particular embodiments only and is not intended to be limiting of the invention. As used herein, for example, the singular forms “a.” “an,” and “the” are intended to include the plural forms as well, unless the context clearly indicates otherwise. It will be further understood that the terms “contains”, “containing”, “includes”, “including.” “comprises”, and/or “comprising.” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation are used herein merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection, and are not limited to either unless expressly referenced as such.

As used herein, the term “cement nodules” refers to the hardened and welded clastic sediments formed by the precipitation of mineral matter in pore spaces. Cement nodules may be a form of nodular evaporite deposits.

As used herein, “clastic rock” or “clastic sediment” refers to a rock composed of fragments (clasts) of pre-existing minerals and rock and includes sandstone, siltstone, or claystone.

As used herein, “degree of occlusion” refers to the amount of occluded space in the rock pores. The degree of occlusion may be estimated by visually comparing cemented and non-cemented areas of the rock to determine a color or property change. Other scientific techniques may include log- or core-based porosity calculations or direct rock lab-measurements.

As used herein, “macro-nodule” refers to a cement nodule greater than 1 cm in diameter.

As used herein, “meso-nodule” refers to a cement nodule about 1 mm to about 1 cm in diameter.

As used herein, “micro-nodule” refers to a cement nodule smaller than 1 mm in diameter.

As used herein, “non-cemented” refers to diagenetic rock facies containing no detectable cement nodules and will thus have no diagenetic imprint on the reservoir capacity or performance.

As used herein, “weakly cemented” refers to diagenetic rock facies with greater than 0 vol % and up to about 3 vol % of cement nodules based on the total volume of the diagenetic rock and will thus have a weak diagenetic imprint on the reservoir capacity and performance.

As used herein, “moderately cemented” refers to diagenetic rock facies with greater than about 3 vol % and up to about 10 vol % of cement nodules based on the total volume of the diagenetic rock and will thus have a considerable diagenetic imprint on the reservoir capacity and performance.

As used herein, “severely cemented” refers to diagenetic rock facies with greater than 10 vol % of cement nodules based on the total volume of the diagenetic rock and will thus have a destructive diagenetic imprint on the reservoir capacity and performance.

As used herein, “roundness index” refers to the sphericity of an individual cement nodule and is calculated as the ratio of the length of two or more axes of the nodule.

Methods of classifying the diagenetic rock facies of subterranean formations are described herein. For example, a subterranean formation may be imaged using one or more conventional methods. Image analysis may be used to identify and characterize nodular evaporite deposits in subterranean formations from the formation images. The diagenetic rock facies of the formations may be classified according to the determined characteristics of the nodular evaporite deposits. From the classifications, a 3D model of the diagenetic rock facies of the subterranean formation may be generated to aid in the selection of future petroleum exploration and production sites.

The subterranean formations of the present disclosure may comprise clastic (also referred to as siliciclastic) reservoirs and/or non-clastic reservoirs. Examples of clastic reservoir rocks may include, but are not limited to, sandstone, mudrock, breccia, conglomerates, the like, and any combination thereof. Conversely, examples of non-clastic reservoir rocks may include, but are not limited to, carbonates, limestone, dolomites, travertine, the like, and any combination thereof.

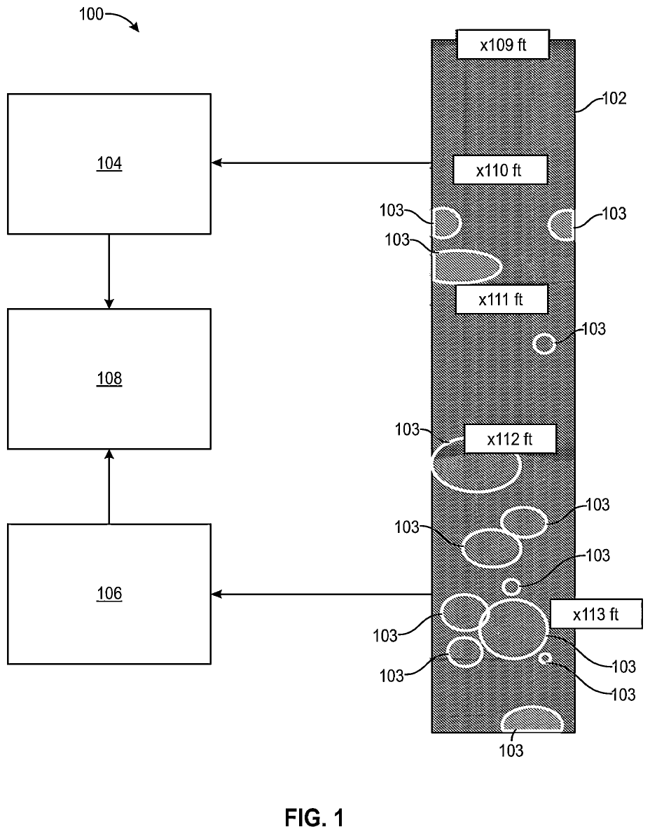

FIG. 1 illustrates a non-limiting example of a diagenetic rock facies classification method 100. A formation image 102 is obtained by imaging a portion of a subterranean formation. The formation image 102 is analyzed to identify a number 104 of one or more nodular evaporite deposits 103 per unit volume. Additionally, one or more deposit characteristics 106 are determined for each of the one or more nodular evaporite deposits 103. Examples of deposit characteristics may include, but are not limited to, cross-sectional area, volume, roundness index, vertical depth relative to the subterranean formation, degree of occlusion, overlap of deposits, the like, and any combination thereof. The diagenetic rock facies 108 of the subterranean formation is classified based on the number 104 of one or more nodular evaporite deposits 103 per unit volume and the one or more deposit characteristics 106 of each of the one or more nodular evaporite deposits 103 per unit volume.

Portions of the subterranean formations may be in situ or core samples, and the portions of the subterranean formations may be characterized through various image analyses. The source of the formation images may be from conventional petroleum exploration and production procedures. Procedures used to image core samples, such as 360° core photography and/or a slabbed core photography, may be used to obtain a 360° core photo or slabbed core photo, respectively. Well logging techniques may also be used to image an in situ sample (e.g., a portion of a borchole). Examples of suitable well logs include, but are not limited to, borchole image logs, resistivity logs, density logs, sonic logs, neutron capture gamma-ray spectroscopy logs, the like, and any combination thereof.

The formation images of the portion of the subterranean formation may require digital processing to improve the quality of the images. For example, digital image processing methods such as filtering, affine transformations, image denoising, recoloring, or any combination thereof may be used to enhance formation images. Image enhancement may aid in identifying nodular evaporite deposits from the formation background.

Individual nodular evaporite deposits may be identified from the formation image manually (e.g., by eye) and/or digitally. Digital identification processes may be integrated into conventional well logging software for a formation image. Techniques such as machine learning and artificial intelligence may be used to enhance the digital identification processes. Identification processes (manual and/or digital) may be used to glean physical properties of the individual nodular evaporite deposits, including, but not limited to, the deposits' length, width, the like, and any combination thereof.

The nodular evaporite deposits identified in the subterranean formations may, for example, comprise cement nodules and/or occluded spaces. Furthermore, nodular evaporite deposits may be at least partially composed of or wholly composed of minerals that include, but not limited to, anhydrite, calcite, halite, sylvite, carnallite, langbeinite, polyhalite, kainite, the like, and any combination thereof.

One or more deposit characteristics of the nodular evaporite deposits may be calculated from the physical properties extracted from the formation images. Examples of specific nodular evaporite deposit characteristics include, but are not limited to, cross-sectional area, volume, roundness index, vertical depth relative to the subterranean formation, degree of occlusion, overlap of deposits, the like, and any combination thereof.

The nodular evaporite deposits may, for example, have a cross-sectional area of about 0.01 mm² to about 100 cm² (or about 0.01 mm² to about 1 cm², or about 0.1 mm² to about 10 cm², or about 1 mm² to about 10 cm²). Further, the solid volume (i.e., the volume of a nodular evaporite deposit not including the volume of the voids) of the nodular evaporite deposits may, for example, be about 0.001 mm³ to about 5000 cm³ (or about 0.001 mm³ to about 100 cm³, or about 1 cm³ to about 1000 cm³, or about 100 cm³ to about 5000 cm³).

The frequency of the nodular evaporite deposits' occurrence may, for example, be about 1 deposit per meter depth to about 100 deposits per meter depth (or about 1 deposit per meter depth to about 25 deposits per meters depth, or about 10 deposits per meter depth to about 50 deposits per meters depth, or about 25 deposits per meter depth to about 75 deposits per meters depth, or about 50 deposits per meter depth to about 100 deposits per meters depth). The frequency of the deposits may be such that the entire portion of the subterranean formation is occluded by the deposits.

Individual nodular evaporite deposits may occur as single nodule or overlap with one or more additional nodular evaporite deposits.

One or more of the above characteristics of the nodular evaporite deposits may be calibrated using geochemical logs that measure the composition of the subterranean formation rock. For example, the volume of individual nodular evaporite deposits calculated from the formation images may be compared to the volume of the same nodular evaporite deposits determined by geochemical logs, such as a neutron capture gamma-ray spectroscopy log.

Once characterized, one or more of the nodular evaporite deposits, if determined to be cement nodules, may be defined as micro-nodules, meso-nodules, or macro-nodules. Specifically, micro-nodules and meso-nodules may be defined from the characteristics determined from formation images of core samples, while formation images comprising well logs may be used to determine the characteristics that define meso-nodules and macro-nodules.

The presence of one or more micro-nodules, meso-nodules, or macro-nodules per unit volume in the subterranean formation and/or the nodules' characteristics may be used to classify the diagenetic rock facies of the formation. For example, the diagenetic rock facies may be classified as non-cemented (e.g., a subterranean formation lacking cement nodules), weakly cemented, moderately cemented, or severely cemented. Additionally, the diagenetic rock facies of the subterranean formation may be classified according to one or more other existing classification methods.

The classification of the diagenetic rock facies of the subterranean formation may be used to infer one or more additional geophysical attributes of the formation rock including, but not limited to, petroleum capacity, permeability, the like, and any combination thereof. Further, the inferred geophysical attributes may be contributed to a 3D model of the subterranean formation. The geographical location of future petroleum exploration and production sites may be selected given the 3D model of the subterranean formation.

The capacity and performance of a reservoir in the subterranean formation may be verified using methods including, but not limited to, in-situ reservoir flow/pressure tests, wireline-logging techniques such as density logs or neutron logs, surface flow tests, the like, and any combination thereof. The performance of such tests may correlate the flow of hydrocarbons to the diagenetic facies of the rock.

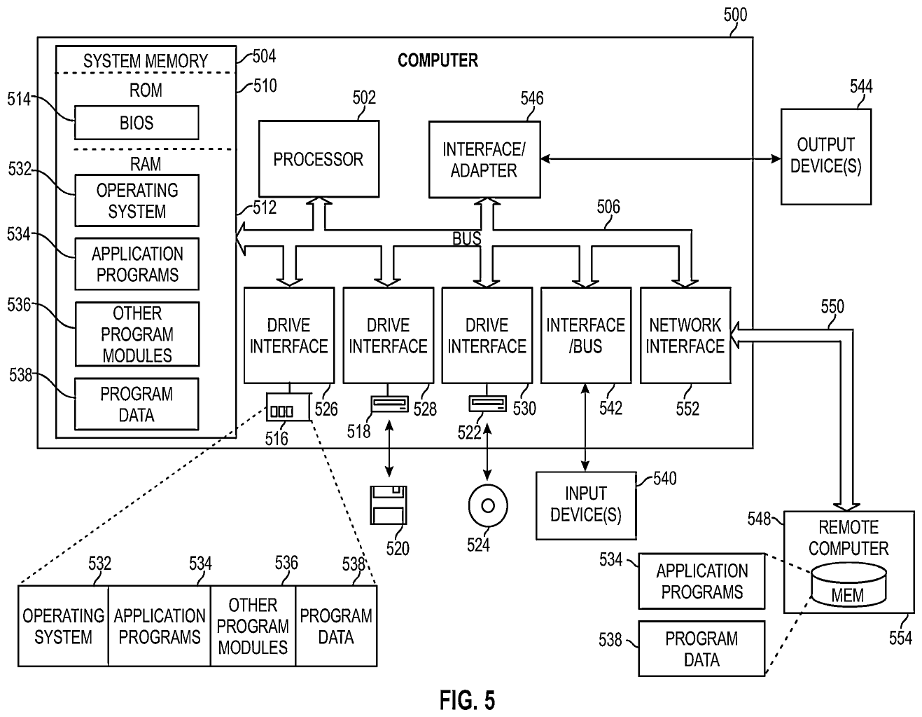

In view of the foregoing structural and functional description, those skilled in the art will appreciate that portions of the embodiments may be embodied as a method, data processing system, or computer program product. Accordingly, these portions of the present embodiments may take the form of an entirely hardware embodiment, an entirely software embodiment, or an embodiment combining software and hardware, such as shown and described with respect to the computer system of FIG. 5. Furthermore, portions of the embodiments may be a computer program product on a computer-usable storage medium having computer readable program code on the medium. Any non-transitory, tangible storage media possessing structure may be utilized including, but not limited to, static and dynamic storage devices, hard disks, optical storage devices, and magnetic storage devices, but excludes any medium that is not eligible for patent protection under 35 U.S.C. § 101 (such as a propagating electrical or electromagnetic signals per se). As an example and not by way of limitation, computer-readable storage media may include a semiconductor-based circuit or device or other IC (such, as for example, a field-programmable gate array (FPGA) or an ASIC), a hard disk, an HDD, a hybrid hard drive (HHD), an optical disc, an optical disc drive (ODD), a magneto-optical disc, a magneto-optical drive, a floppy disk, a floppy disk drive (FDD), magnetic tape, a holographic storage medium, a solid-state drive (SSD), a RAM-drive, a SECURE DIGITAL card, a SECURE DIGITAL drive, or another suitable computer-readable storage medium or a combination of two or more of these, where appropriate. A computer-readable non-transitory storage medium may be volatile, nonvolatile, or a combination of volatile and non-volatile, as appropriate.

Certain embodiments have also been described herein with reference to block illustrations of methods, systems, and computer program products. It will be understood that blocks and/or combinations of blocks in the illustrations, as well as methods or steps or acts or processes described herein, can be implemented by a computer program comprising a routine of set instructions stored in a machine-readable storage medium as described herein. These instructions may be provided to one or more processors of a general purpose computer, special purpose computer, or other programmable data processing apparatus (or a combination of devices and circuits) to produce a machine, such that the instructions of the machine, when executed by the processor, implement the functions specified in the block or blocks, or in the acts, steps, methods and processes described herein.

These processor-executable instructions may also be stored in computer-readable memory that can direct a computer or other programmable data processing apparatus to function in a particular manner, such that the instructions stored in the computer-readable memory result in an article of manufacture including instructions which implement the function specified. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

In this regard, FIG. 5 illustrates one example of a computer system 500 that can be employed to execute one or more embodiments of the present disclosure. Computer system 500 can be implemented on one or more general purpose networked computer systems, embedded computer systems, routers, switches, server devices, client devices, various intermediate devices/nodes or standalone computer systems. Additionally, computer system 500 can be implemented on various mobile clients such as, for example, a personal digital assistant (PDA), laptop computer, pager, and the like, provided it includes sufficient processing capabilities.

Computer system 500 includes processing unit 502, system memory 504, and system bus 506 that couples various system components, including the system memory 504, to processing unit 502. Dual microprocessors and other multi-processor architectures also can be used as processing unit 502. System bus 506 may be any of several types of bus structure including a memory bus or memory controller, a peripheral bus, and a local bus using any of a variety of bus architectures. System memory 504 includes read only memory (ROM) 510 and random access memory (RAM) 512. A basic input/output system (BIOS) 514 can reside in ROM 510 containing the basic routines that help to transfer information among elements within computer system 500.

Computer system 500 can include a hard disk drive 516, magnetic disk drive 518, e.g., to read from or write to removable disk 520, and an optical disk drive 522, e.g., for reading CD-ROM disk 524 or to read from or write to other optical media. Hard disk drive 516, magnetic disk drive 518, and optical disk drive 522 are connected to system bus 506 by a hard disk drive interface 526, a magnetic disk drive interface 528, and an optical drive interface 530, respectively. The drives and associated computer-readable media provide nonvolatile storage of data, data structures, and computer-executable instructions for computer system 500. Although the description of computer-readable media above refers to a hard disk, a removable magnetic disk and a CD, other types of media that are readable by a computer, such as magnetic cassettes, flash memory cards, digital video disks and the like, in a variety of forms, may also be used in the operating environment; further, any such media may contain computer-executable instructions for implementing one or more parts of embodiments shown and described herein.

A number of program modules may be stored in drives and RAM 510, including operating system 532, one or more application programs 534, other program modules 536, and program data 538. In some examples, the application programs 534 can include image analysis programs; and the program data 538 can include the formation images. The application programs 534 and program data 538 can include functions and methods programmed to perform various analyses or apply various algorithms described herein.

A user may enter commands and information into computer system 500 through one or more input devices 540, such as a pointing device (e.g., a mouse, touch screen), keyboard, microphone, joystick, game pad, scanner, and the like. These and other input devices 540 are often connected to processing unit 502 through a corresponding port interface 542 that is coupled to the system bus, but may be connected by other interfaces, such as a parallel port, serial port, or universal serial bus (USB). One or more output devices 544 (e.g., display, a monitor, printer, projector, or other type of displaying device) is also connected to system bus 506 via interface 546, such as a video adapter.

Computer system 500 may operate in a networked environment using logical connections to one or more remote computers, such as remote computer 548. Remote computer 548 may be a workstation, computer system, router, peer device, or other common network node, and typically includes many or all the elements described relative to computer system 500. The logical connections, schematically indicated at 550, can include a local area network (LAN) and/or a wide area network (WAN), or a combination of these, and can be in a cloud-type architecture, for example configured as private clouds, public clouds, hybrid clouds, and multi-clouds. When used in a LAN networking environment, computer system 500 can be connected to the local network through a network interface or adapter 552. When used in a WAN networking environment, computer system 500 can include a modem, or can be connected to a communications server on the LAN. The modem, which may be internal or external, can be connected to system bus 506 via an appropriate port interface. In a networked environment, application programs 534 or program data 538 depicted relative to computer system 500, or portions thereof, may be stored in a remote memory storage device 554.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

Embodiments disclosed herein include the following

Embodiment A. A method including imaging a portion of a subterranean formation to obtain a formation image; analyzing the formation image to identify one or more nodular evaporite deposits in the portion of the subterranean formation; determining one or more deposit characteristics for each of the one or more nodular evaporite deposits, the one or more deposits characteristics comprising a cross-sectional area, a volume, a roundness index, a vertical depth relative to the subterranean formation, a degree of occlusion, an overlap of deposits, or any combination thereof; and classifying the portion of the subterranean formation into a diagenetic rock facies based on the one or more deposit characteristics of each of the one or more nodular evaporite deposits per unit volume and a number of the one or more nodular evaporite deposits per the unit volume.

Embodiment B. A method including imaging a portion of a subterranean formation to obtain a formation image; analyzing the formation image to identify one or more nodular evaporite deposits in the portion of the subterranean formation; determining one or more deposit characteristics for each of the one or more nodular evaporite deposits, the one or more deposits characteristics comprising a cross-sectional area, a volume, a roundness index, a vertical depth relative to the subterranean formation, a degree of occlusion, an overlap of deposits, or any combination thereof; classifying the portion of the subterranean formation into a diagenetic rock facies based on the one or more deposit characteristics of each of the one or more nodular evaporite deposits per unit volume and a number of the one or more nodular evaporite deposits per the unit volume; and generating a 3D model of the portion of the subterranean formation from the diagenetic rock facies.

Each of Embodiments A and B may have one or more of the following additional elements in any combination: Element 1: wherein the diagenetic rock facies are classified as non-cemented, weakly cemented, moderately cemented, or severely cemented. Element 2: wherein the portion of the subterranean formation is a core sample; and wherein the formation image comprises a 360° core photo and/or a slabbed core photograph. Element 3: wherein the portion of the subterranean formation is in situ; and wherein the formation image comprises a well log. Element 4: Element 3 and wherein the well log comprises a borehole image log, a resistivity log, a density log, a sonic log, a neutron capture gamma-ray spectroscopy log, or any combination thereof. Element 5: wherein the one or more nodular evaporite deposits comprise an occluded space, a cement nodule, or any combination thereof. Element 6: Element 5 and wherein the cement nodule comprises a meso-nodule, a macro-nodule, a micro-nodule, or any combination thereof. Element 7: The method of claim 1, wherein the one or more nodular evaporite deposits comprise anhydrite, calcite, halite, sylvite, carnallite, langbeinite, polyhalite, kainite, or any combination thereof. Element 8: further comprising calibrating the one or more deposit characteristics with data obtained from geochemical logs. Element 9: wherein the subterranean formation is part of a clastic reservoir. Element 10: further comprising selecting a geographical location of future petroleum exploration and production sites based on the 3D model of the portion of the subterranean formation.

By way of non-limiting example, exemplary combinations applicable to Embodiments A and B include: Element 1 in combination with one or more of Elements 2-10; Element 2 in combination with one or more of Elements 3-10; Element 3 (optionally in combination with Element 4) in combination with one or more of Elements 5-10; Element 5 (optionally in combination with Element 6) in combination with one or more of Elements 7-10; and two or more of Elements 7-10 in combination.

### Examples

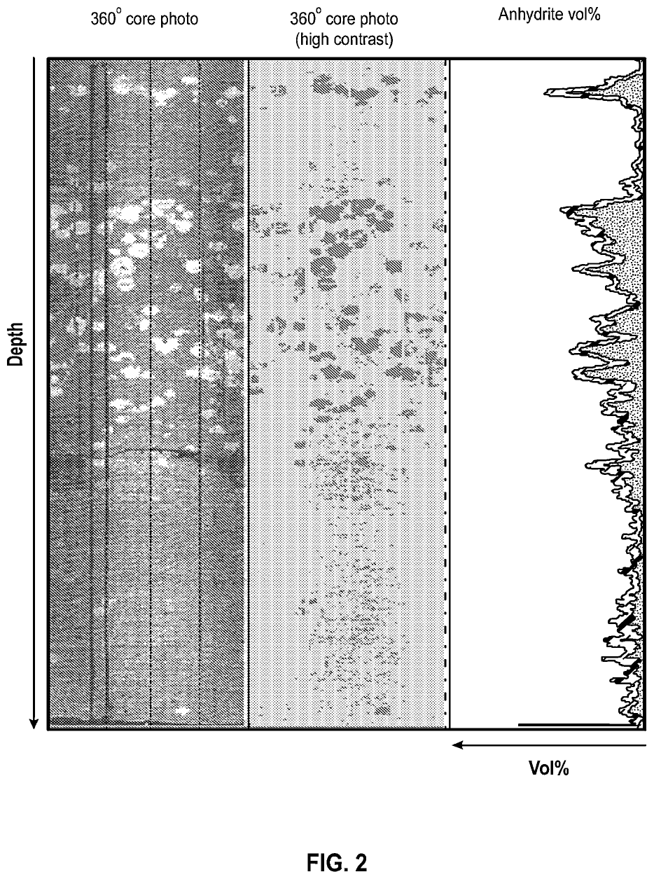

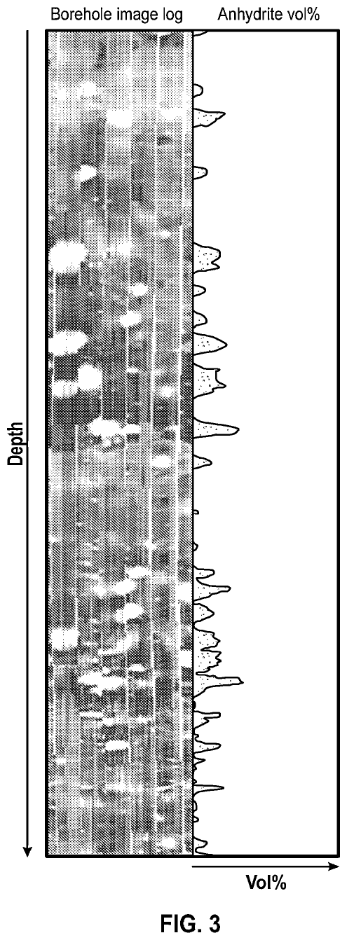

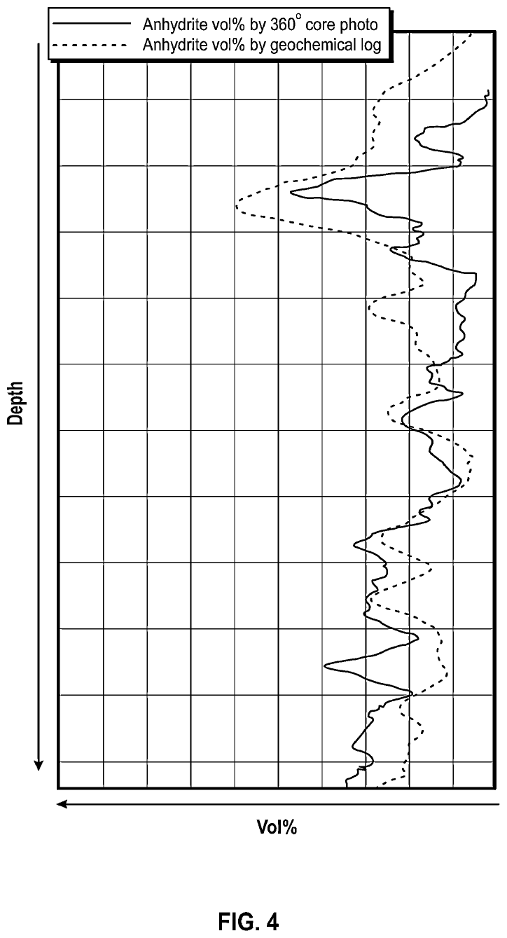

The methods of the present disclosure were used to identify and characterize the nodular evaporite deposits present in a portion of a subterranean formation. FIG. 2 is a 360° core photo taken of a core sample containing nodular evaporite deposits comprising anhydrite (left panel). The 360° core photo was recolored to improve the image contrast between the nodular evaporite deposits and the background core sample (FIG. 2, middle panel). From the high contrast 360° core photo, the length and width of the individual nodular evaporite deposits were extracted. The lengths and widths of the deposits were then used to calculate the percent volume of anhydrite relative to the total core sample volume (FIG. 2, right panel). Similarly, borehole image logs were obtained of a borehole in a subterranean formation containing evaporite nodular evaporite deposits comprising anhydrite (FIG. 3, left panel). The length and width of each nodular evaporite deposit was measured from the borehole image log, from which the percent volume of anhydrite relative to the borchole volume was calculated (FIG. 3, right panel). Finally, the calculated anhydrite percent volumes were further compared to the anhydrite percent volumes determined by a geochemical log (FIG. 4). FIG. 3 confirms that the percent volume of anhydrite calculated using the methods of the present disclosure correlate strongly to the geochemical log data.

The invention claimed is:

1. A method comprising:
imaging a portion of a subterranean formation to obtain a formation image;
analyzing the formation image to identify one or more nodular evaporite deposits in the portion of the subterranean formation;
determining one or more deposit characteristics for each of the one or more nodular evaporite deposits, the one or more deposit characteristics comprising a cross-sectional area, a volume, a roundness index, a vertical depth relative to the subterranean formation, a degree of occlusion, an overlap of deposits, or any combination thereof; and
classifying the portion of the subterranean formation into a diagenetic rock facies based on the one or more deposit characteristics of each of the one or more nodular evaporite deposits per unit volume and a number of the one or more nodular evaporite deposits per the unit volume.

2. The method of claim 1, wherein the diagenetic rock facies are classified as non-cemented, weakly cemented, moderately cemented, or severely cemented.

3. The method of claim 1, wherein the portion of the subterranean formation is a core sample; and wherein the formation image comprises a 360° core photo and/or a slabbed core photograph.

4. The method of claim 1, wherein the portion of the subterranean formation is in situ; and
wherein the formation image comprises a well log.

5. The method of claim 4, wherein the well log comprises a borehole image log, a resistivity log, a density log, a sonic log, a neutron capture gamma-ray spectroscopy log, or any combination thereof.

6. The method of claim 1, wherein the one or more nodular evaporite deposits comprise an occluded space, a cement nodule, or any combination thereof.

7. The method of claim 6, wherein the cement nodule comprises a meso-nodule, a macro-nodule, a micro-nodule, or any combination thereof.

8. The method of claim 1, wherein the one or more nodular evaporite deposits comprise anhydrite, calcite, halite, sylvite, carnallite, langbeinite, polyhalite, kainite, or any combination thereof.

9. The method of claim 1, further comprising calibrating the one or more deposit characteristics with data obtained from geochemical logs.

10. The method of claim 1, wherein the subterranean formation is part of a clastic reservoir.

11. A method comprising:
imaging a portion of a subterranean formation to obtain a formation image;
analyzing the formation image to identify one or more nodular evaporite deposits in the portion of the subterranean formation;
determining one or more deposit characteristics for each of the one or more nodular evaporite deposits, the one or more deposit characteristics comprising a cross-sectional area, a volume, a roundness index, a vertical depth relative to the subterranean formation, a degree of occlusion, an overlap of deposits, or any combination thereof;
classifying the portion of the subterranean formation into a diagenetic rock facies based on the one or more deposit characteristics of each of the one or more nodular evaporite deposits per unit volume and a number of the one or more nodular evaporite deposits per the unit volume; and
generating a 3D model of the portion of the subterranean formation from the diagenetic rock facies.

12. The method of claim 11, further comprising selecting a geographical location of future petroleum exploration and production sites based on the 3D model of the portion of the subterranean formation.

13. The method of claim 11, wherein the diagenetic rock facies are classified as non-cemented, weakly cemented, moderately cemented, or severely cemented.

14. The method of claim 11, wherein the portion of the subterranean formation is a core sample; and wherein the formation image comprises a 360° core photo and/or a slabbed core photograph.

15. The method of claim 11, wherein the portion of the subterranean formation is in situ; and wherein the formation image comprises a well log.

16. The method of claim 15, wherein the well log comprises a borehole image log, a resistivity log, a density log, a sonic log, a neutron capture gamma-ray spectroscopy log, or any combination thereof.

17. The method of claim 11, wherein the one or more nodular evaporite deposits comprise an occluded space, a cement nodule, or any combination thereof.

18. The method of claim 17, wherein the cement nodule comprises a meso-nodule, a macro-nodule, a micro-nodule, or any combination thereof.

19. The method of claim 11, wherein the one or more nodular evaporite deposits comprise anhydrite, calcite, halite, sylvite, carnallite, langbeinite, polyhalite, kainite, or any combination thereof.

20. The method of claim 11, further comprising calibrating the one or more deposit characteristics with data obtained from geochemical logs.
